(19) United States
(12) Patent Application Publication
(10) Pub. No.: US 2025/0101002 A1
(43) Pub. Date: Mar. 27, 2025

(54) DEUTERATED INFIGRATINIB COMPOUNDS

(71) Applicant: QED Therapeutics, Inc., Palo Alto, CA (US)

(72) Inventor: Robert Zamboni, San Francisco, CA (US)

(21) Appl. No.: 18/599,623

(22) Filed: Mar. 8, 2024

Related U.S. Application Data
(60) Provisional application No. 63/489,168, filed on Mar. 8, 2023.

(51) Int. Cl. C07D 403/12; A61K 31/506; C07B 59/00

(52) U.S. Cl. CPC C07D 403/12; A61K 31/506; C07B 59/004

(57) ABSTRACT

Provided herein are compounds, or salts (e.g., pharmaceutically acceptable salts), esters, tautomers, prodrugs, zwitterionic forms, or stereoisomers thereof, which compounds comprise one or more deuterium atoms, as well as pharmaceutically compositions comprising the same. Also provided herein are methods of using the same in the treatment of various diseases, disorders, and conditions including cancers and skeletal dysplasias.

### CROSS REFERENCE TO RELATED APPLICATIONS

This application claims the benefit of and priority to U.S. Patent Application No. 63/489,168, filed on Mar. 8, 2023, the entire contents of which are incorporated by reference herein.

### BACKGROUND

Fibroblast growth factors (FGFs) are cell signaling proteins that are crucial to development in animal cells. The human FGF family includes 23 structurally related signaling molecules, several of which are known to bind fibroblast growth factor receptors (FGFRs). The mammalian FGFR family includes 4 members, FGFR1, FGFR2, FGFR3, and FGFR4. FGFRs comprise an extracellular ligand binding domain, a transmembrane domain, and an intracellular tyrosine kinase domain that undergoes phosphorylation upon binding an FGF.

The FGF/FGFR signaling pathway is implicated in a variety of cancers and other disorders. FGFR mutations such as FGFR1 and FGFR2 mutations, including FGFR gene fusions and rearrangements, are linked to various congenital malformations of the musculoskeletal system including Kallmann syndrome, hypogonadotropic hypogonadism, cleft palate and other craniofacial defects, scoliosis, Type 1 or classic Pfeiffer syndrome, osteoglophonic dysplasia, craniosynostosis, mandibular progrnathism, hypertelorism, brachydactyly, inter-phalangeal joint fusion, Jackson-Weiss syndrome, Antley-Bixler syndrome, Apert syndrome, Crouzon syndrome, and trigonocephaly. FGFR mutations have also been linked to various cancers including lung, breast, head and neck, urinary bladder, endometrial, prostate, ovarian, colorectal, salivary gland, and hematological cancers as well as phosphaturic mesenchymal tumors, rhabdomyosarcoma, sarcomas, melanomas, and glioblastomas. In particular, mutations in FGFR3 including FGFR3 gene fusions and rearrangements have been linked to epithelial, bladder, and cervix cancers, urothelial carcinoma, cholangiocarcinoma, glioblastoma, and myelomas. Mutations in FGFRs such as FGFR3, including FGFR gene fusions and rearrangements, are also linked to skeletal dysplasias and craniosynostoses including achondroplasia, hypochondroplasia, thanatophoric dysplasia severe achondroplasia with developmental delay and acanthosis nigricans (SADDAN), Muenke syndrome, and Crouzon syndrome (e.g., Crouzon syndrome acanthosis nigricans). Accordingly, inhibition of FGFRs has emerged as a potential strategy for treating various cancers and skeletal dysplasias.

Skeletal dysplasias can affect bone development, cartilage growth, and neurological functioning. Certain skeletal dysplasias, including achondroplasia (ACH) and hypochondroplasia (HCH), are forms of dwarfism. Achondroplasia (ACH) is the most common nonlethal form of skeletal dysplasia, affecting between 1 in 15,000 to 1 in 30,000 individuals (Horton W A, Hall J G, Hecht J T., Achondroplasia. Lancet, 2007, 370, 162-72; Waller D K, Correa A, Vo T M, Wang Y, Hobbs C, Langlois P H, et al., US. Am. J. Med. Genet. A., 2008, 146A (18), 2385-2389). People with ACH have short stature, on average growing to approximately 4 feet (˜125 cm) in height, and are prone to significant co-morbidities including sleep apnea, chronic otitis media with conductive hearing loss, spinal stenosis, obesity, and in some cases, narrowing of the foramen magnum that can require urgent surgical intervention. HCH is similar to ACH and is characterized by less pronounced physical signs; however, HCH patients may also be impacted by greater neurological challenges including mild to moderate intellectual disabilities or learning problems.

While various therapies have been explored for cancers and skeletal dysplasias including achondroplasia, such therapies are generally not curative and can be burdensome on patients. Therefore, there remains an unmet need to develop therapeutic strategies for treating cancers and skeletal dysplasias.

### SUMMARY

The present disclosure provides compounds, as well as compositions and kits comprising the same, and methods of using the same in the treatment of diseases and disorders such as cancers. The present disclosure provides compounds that may be capable of inhibiting an FGFR such as one or more of FGFR1, FGFR2, FGFR3, and FGFR4. In some embodiments, the compounds provided herein are useful in the treatment of cancers and/or skeletal dysplasias, such as cancers and/or skeletal dysplasias characterized by a mutant, rearranged, or fused FGFR. The compounds provided herein include deuterated infigratinib compounds; that is, infigratinib that is substituted with one or more deuterium atoms.

In an aspect, the present disclosure provides compositions comprising compounds represented by Formula I:

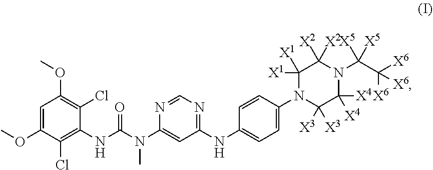

in which each X¹, X², X³, X⁴, X⁵, and X⁶ is independently selected from hydrogen and deuterium (e.g., as described herein), or a salt (e.g., pharmaceutically acceptable salt), ester, tautomer, prodrug, zwitterionic form, or stereoisomer thereof, provided that at least one X¹, X², X³, X⁴, X⁵, or X⁶ is a deuterium. In some embodiments, the compound is represented by Formula Ia, Ib, Ic, Id, Ie, or If:

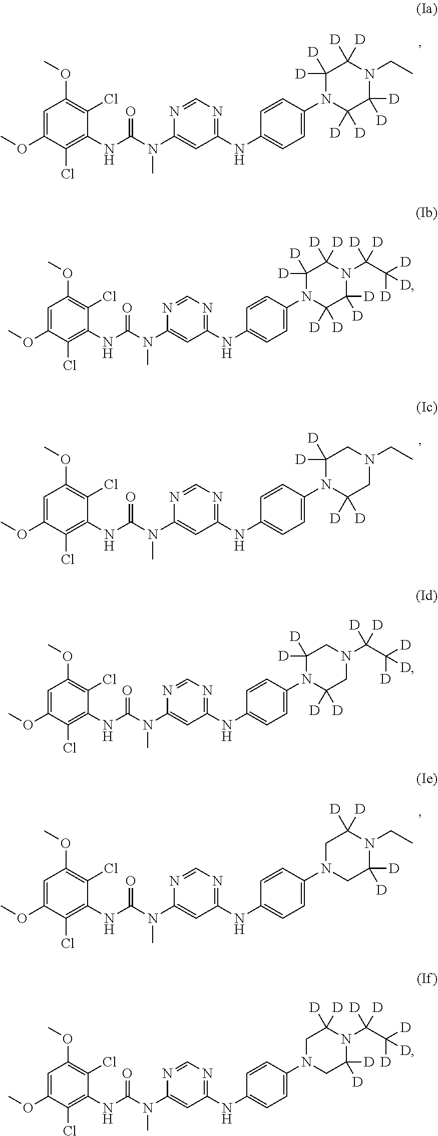

or a salt (e.g., pharmaceutically acceptable salt), ester, tautomer, prodrug, zwitterionic form, or stereoisomer thereof. In some embodiments, a compound provided herein, or a salt, ester, tautomer, zwitterionic form, or stereoisomer thereof, can modulate (e.g., inhibit) the activity of an FGFR, such as FGFR1, FGFR2, FGFR3, and/or FGFR4. In some embodiments, a compound provided herein, or a salt, ester, tautomer, zwitterionic form, or stereoisomer thereof, can selectively inhibit the activity of FGFR1, FGFR2, FGFR3, and/or FGFR4.

In another aspect, the present disclosure provides a pharmaceutical composition comprising a compound provided herein (e.g., a compound represented by any one of Formulas I, Ia, Ib, Ic, Id, Ie, and If, or any other formula set forth herein), or a salt, ester, tautomer, prodrug, zwitterionic form, or stereoisomer thereof, together with a pharmaceutically acceptable carrier.

In another aspect, the present disclosure provides a method of inhibition of FGFR (e.g., FGFR1, FGFR2, FGFR3, and/or FGFR4) activity in a cell using, e.g., a compound provided herein (e.g., a compound represented by any one of Formulas I, Ia, Ib, Ic, Id, Ie, and If, or any other formula set forth herein), or a salt, ester, tautomer, prodrug, zwitterionic form, or stereoisomer thereof, or a pharmaceutical composition comprising the same. In some embodiments, the cell is located within a subject (e.g., a human subject).

In a further aspect, the present disclosure provides a method of inhibition of FGFR (e.g., FGFR1, FGFR2, FGFR3, and/or FGFR4) activity in a human or animal subject for the treatment of a disease, disorder, or condition such as a cancer or skeletal dysplasia using, e.g., a compound provided herein (e.g., a compound represented by any one of Formulas I, Ia, Ib, Ic, Id, Ie, and If, or any other formula set forth herein), or a salt, ester, tautomer, prodrug, zwitterionic form, or stereoisomer thereof, or a pharmaceutical composition comprising the same.

In a related aspect, the present disclosure provides a method of treating a disease, disorder, or condition such as a cancer or skeletal dysplasia using, e.g., a compound provided herein (e.g., a compound represented by any one of Formulas I, Ia, Ib, Ic, Id, Ie, and If, or any other formula set forth herein), or a salt, ester, tautomer, prodrug, zwitterionic form, or stereoisomer thereof, or a pharmaceutical composition comprising the same. In some embodiments, the disease, disorder, or condition is a cancer. In some embodiments, the disease, disorder, or condition is a skeletal dysplasia.

In another aspect, the present disclosure provides a use of a compound provided herein (e.g., a compound represented by any one of Formulas I, Ia, Ib, Ic, Id, Ie, and If, or any other formula set forth herein), or a salt, ester, tautomer, prodrug, zwitterionic form, or stereoisomer thereof, in the manufacture of a medicament for the treatment of a disease, disorder, or condition (e.g., a cancer or skeletal dysplasia, as described herein) ameliorated, treated, inhibited, or reduced by inhibition of an FGFR (e.g., FGFR1, FGFR2, FGFR3, and/or FGFR4), including an FGFR comprising one or more mutations or gene fusions. In some embodiments, the disease, disorder, or condition is a cancer. In some embodiments, the disease, disorder, or condition is a skeletal dysplasia.

In a further aspect, the present disclosure provides a compound as provided herein (e.g., a compound represented by any one of Formulas I, Ia, Ib, Ic, Id, Ie, and If, or any other formula set forth herein), or a salt, ester, tautomer, prodrug, zwitterionic form, or stereoisomer thereof, for use as a medicament. In some embodiments, the medicament is used in the treatment of a disease, disorder, or condition (e.g., a cancer or skeletal dysplasia). In some embodiments, the disease, disorder, or condition is a cancer. In some embodiments, the disease, disorder, or condition is a skeletal dysplasia.

### DETAILED DESCRIPTION

The present disclosure provides compounds (e.g., compounds of any one of Formulas I, Ia, Ib, Ic, Id, Ie, and If), which compounds may possess useful FGFR inhibitory activity, and may be used in the treatment or prophylaxis of a disease, disorder, or condition in which FGFR plays an active role. In particular, certain compounds provided herein may possess useful inhibitory activity of an FGFR selected from FGFR1, FGFR2, FGFR3, FGFR4, and a combination thereof. The present disclosure also provides pharmaceutical compositions comprising one or more compounds provided herein together with a pharmaceutically acceptable carrier, as well as methods of making and using the compounds and compositions. The present disclosure also provides methods for inhibiting an FGFR, including a mutated, fused, or rearranged FGFR (e.g., FGFR1, FGFR2, FGFR3, and/or FGFR4). In an aspect, the present disclosure provides a method for treating a disease, disorder, or condition associated with an FGFR mutation, gene fusion, or rearrangement in a subject in need thereof, which method comprises administering to the subject a therapeutically effective amount of a compound or composition provided herein. In some instances, such a disease, disorder, or condition is a cancer. In other instances, such a disease, disorder, or condition is a skeletal dysplasia. Also provided herein is the use of certain compounds provided herein in the manufacture of a medicament for the treatment of a disease, disorder, or condition ameliorated, treated, inhibited, or reduced by inhibition of an FGFR, including a mutated, fused, or rearranged FGFR (e.g., FGFR1, FGFR2, FGFR3, and/or FGFR4). In some instances, such a disease, disorder, or condition is a cancer. In other instances, such a disease, disorder, or condition is a skeletal dysplasia.

When ranges of values are disclosed, and the notation “from n1 . . . to n2” or “between n1 . . . and n2” is used, where n1 and n2 are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

“Bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

Asymmetric centers may exist in the compounds disclosed herein. These centers are designated by the symbols “R” or “S.” depending on the configuration of substituents around the chiral carbon atom. It should be understood that the disclosure encompasses all stereochemical isomeric forms, including diastereomeric, enantiomeric, atropisomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds provided herein may comprise conformational isomers, which compounds comprise groups that can orient in different conformations in relation to another moiety. Additionally, the compounds disclosed herein can exist in unsolvated as well as solvated forms with pharmaceutically acceptable solvents such as water, ethanol, and the like. In general, the solvated forms are considered equivalent to the unsolvated forms.

“Combination therapy” means the administration of two or more therapeutic agents to treat a therapeutic condition or disorder described in the present disclosure. Such administration encompasses co-administration of these therapeutic agents in a substantially simultaneous manner, such as in a single dose unit (e.g., capsule) having a fixed ratio of active ingredients or in multiple, separate dose units (e.g., capsules) for each active ingredient. In addition, such administration also encompasses use of each type of therapeutic agent in a sequential manner. In either case, the treatment regimen will provide beneficial effects of the drug combination in treating the conditions or disorders described herein.

“Therapeutically effective amount” refers to an amount of a compound or of a pharmaceutical composition useful for treating or ameliorating an identified disease, disorder, or condition, or for exhibiting a detectable therapeutic or inhibitory effect. The exact amounts will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

“Treat,” “treating,” and “treatment” refer to any indicia of success in the treatment or amelioration of an injury, pathology, disease, disorder, or condition, including any objective or subjective parameter such as abatement; remission; diminishing of symptoms or making the injury, pathology, disease, disorder, or condition more tolerable to the patient; slowing in the rate of degeneration or decline; making the final point of degeneration less debilitating; and/or improving a patient's physical or mental well-being. The treatment or amelioration of symptoms can be based on objective or subjective parameters, including the results of a physical examination, neuropsychiatric exams, and/or a psychiatric evaluation. Treatment may also be preemptive in nature; i.e., it may include prevention of a disease, disorder, or condition, prevention of onset of one or more symptoms of a disease, disorder, or condition, and/or prevention of escalation of a disease, disorder, or condition. Prevention of a disease, disorder, or condition may involve complete protection from disease, and/or prevention of disease progression (e.g., to a later stage of the disease, disorder, or condition). For example, prevention of a disease may not mean complete foreclosure of any effect related to the diseases at any level, but instead may mean prevention of the symptoms of a disease, disorder, or condition to a clinically significant or detectable level.

“Patient” or “subject” refers to a living organism suffering from or prone to a disease, disorder, or condition that can be treated by administration of a compound or pharmaceutical composition as provided herein. Non-limiting examples include humans, rats, mice, rabbits, hamsters, guinea pigs, cats, dogs, non-human primates (e.g., monkeys), goats, pigs, sheep, cows, deer, horses, and other non-mammalian animals. Examples of mammals that can be treated by administration of a compound or pharmaceutical composition provided herein include, for example, rodents (e.g., rats, mice, squirrels, guinea pigs, hamsters, etc.), lagomorphs (e.g., rabbits, hares, etc.), primates (e.g., monkeys, apes, etc.), bovines (e.g., cattle), odd-toed ungulates (e.g., horses), even-toed ungulates (e.g., bovines such as cattle, ovine such as sheep, caprine such as goats, porcine such as pigs, etc.), and marsupials (e.g., kangaroo, wallaby, wallaroo, sugar glider, etc.). In some embodiments, the patient or subject is human. In some embodiments, the patient or subject is a companion animal such as a cat or dog. In some embodiments, the patient or subject is a farm animal such as a goat, sheep, cow, pig, or horse. In some embodiments, the patient or subject is an exotic animal such as a primate (e.g., monkey), marsupial (e.g., kangaroo, wallaby, wallaroo, sugar glider, etc.), or a non-domesticated or hybrid cat or dog.

“Composition,” as used herein, is intended to encompass a product comprising the specified ingredients in the specified amounts, as well as any product, which results, directly or indirectly, from combination of the specified ingredients in the specified amounts. By “pharmaceutically acceptable” it is meant the carrier, diluent, or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present disclosure include, but are not limited to, binders, fillers, disintegrants, lubricants, coatings, sweeteners, flavors, and colors. One of skill in the art will recognize that other pharmaceutical excipients are useful in the present disclosure.

The term “prodrug” refers to a compound that is made more active in vivo. Certain compounds disclosed herein may also exist as prodrugs. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug.

The compounds disclosed herein can exist as therapeutically acceptable salts (also referred to herein as “pharmaceutically acceptable salts”). The present disclosure includes compounds provided herein in the form of salts, including acid addition salts. Suitable salts include those formed with both organic and inorganic acids. Such acid addition salts will normally be pharmaceutically acceptable. However, non-pharmaceutically acceptable salts may be of utility in the preparation and purification of the compound in question. Basic addition salts may also be formed and be pharmaceutically acceptable.

The terms “therapeutically acceptable salt” and “pharmaceutically acceptable salt” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

“Jointly therapeutically effective amount” as used herein means the amount at which the therapeutic agents, when given separately (in a chronologically staggered manner, especially a sequence-specific manner) to a warm-blooded animal, especially to a human to be treated, show an (additive, but preferably synergistic) interaction (joint therapeutic effect). Whether this is the case can be determined inter alia by following the blood levels, showing that both compounds are present in the blood of the human to be treated at least during certain time intervals.

“Synergistic effect” as used herein refers to an effect of at least two therapeutic agents: an FGFR inhibitor, as defined herein, and an additional agent, which additional agent may be an agent configured to treat a disease, disorder, or condition or a symptom thereof. The effect can be, for example, slowing the symptomatic progression of a proliferative disease, such as cancer, particularly lung cancer, or symptoms thereof. Analogously, a “synergistically effective amount” refers to the amount needed to obtain a synergistic effect.

“A,” “an,” or “a(n)”, when used in reference to a group of substituents or “substituent group” herein, mean at least one. For example, where a compound is substituted with “an” alkyl or aryl, the compound is unsubstituted or substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

It will be recognized that some variation of natural isotopic abundance occurs in a synthesized compound depending upon the origin of chemical materials used in the synthesis. Thus, a preparation of infigratinib will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention.

For the compounds provided herein, any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Further, unless otherwise stated, when a position is designated specifically as “D” or “deuterium,” the position is understood to have deuterium at an abundance that is at least 3000 times greater than the natural abundance of deuterium, which is 0.015% (i.e., at least 45% incorporation of deuterium).

The term “isotopic enrichment factor” as used herein means the ratio between the isotopic abundance and the natural abundance of a specified isotope.

In some embodiments, a compound provided herein has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75%) deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to a species in which the chemical structure differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in total shall generally be less than 49.9% of the compound. In other embodiments, the relative amount of such isotopologues in total may be less than 47.5%, less than 40%, less than 32.5%, less than 25%, less than 17.5%, less than 10%, less than 5%>, less than 3%>, less than 1%, or less than 0.5% of the compound.

As used herein, the term “substituted with deuterium” refers to the replacement of one or more hydrogen atoms with a corresponding number of deuterium atoms.

### Infigratinib

Infigratinib, as depicted in Formula (II), is a selective and ATP-competitive pan-fibroblast growth factor receptor (FGFR) kinase inhibitor, also known as 3-(2,6-dichloro-3,5-dimethoxyphenyl)-1-{6-[4-(4-ethyl-1-piperazin-1-yl)phenylamino]pyrimidinyl-4-yl}-1-methylurea. Infigratinib selectively inhibits the kinase activity of FGFR1, FGFR2, and FGFR3.

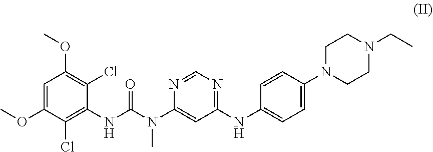 [SMILES: CCN1CCN(c2ccc(Nc3cc(N(C)C(=O)Nc4c(Cl)c(OC)cc(OC)c4Cl)ncn3)cc2)CC1]

Infigratinib is described in U.S. Pat. No. 8,552,002, which is herein incorporated by reference in its entirety. A method of chemically synthesizing infigratinib (including Example 1 provided herein), several crystalline and amorphous forms of infigratinib (including the anhydrous crystalline monophosphate salt described herein), and methods of preparing said forms (including Example 2 provided herein) were described in U.S. Pat. No. 9,067,896, which is herein incorporated by reference in its entirety. The monophosphate salt of infigratinib is alternately referred to herein as BGJ398, infigratinib phosphate, and infigratinib monophosphate.

Infigratinib has been investigated for use in the treatment of various cancers and skeletal dysplasias. Infigratinib monophosphate was approved by the United States Food and Drug Administration (FDA) for use in the treatment of previously treated, unresectable locally advanced or metastatic cholangiocarcinoma. Infigratinib monophosphate has also been studied for use in the treatment of other cholangiocarcinomas, gliomas, glioblastoma, gastric cancers, gastroesophageal junction adenocarcinoma, urothelial carcinomas, bladder cancers, breast cancers, liver cancers, and tumor-induced osteomalacias. Infigratinib monophosphate is also currently in clinical trials for achondroplasia.

For the FDA-approved cholangiocarcinoma indication, infigratinib monophosphate was dosed at 125 milligrams (mg). Two metabolites, BHS697 and CQM157, were determined to contribute between about 16-33% and 9-12% of overall pharmacologic activity, respectively. Common adverse reactions to infigratinib monophosphate included nail toxicity, stomatitis, dry eye, fatigue, alopecia, palmar-plantar erythrodyesthesia syndrome, arthralgia, dysgeusia, constipation, abdominal pain, dry mouth, eyelash changes, diarrhea, dry skin, decreased appetite, blurred vision, and vomiting. Additional adverse reactions including ocular toxicity, hyperphosphatemia and soft tissue mineralization, infections, anemia, pyrexia, abdominal pain, hypercalcemia, and sepsis were also observed in corresponding clinical trials.

Despite the beneficial activities of infigratinib, there remains a need for new compounds to treat cancers and skeletal dysplasias.

### Therapeutic Compounds

In an aspect, the present disclosure provides a compound represented by Formula I:

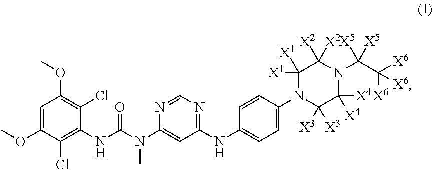

or a salt (e.g., pharmaceutically acceptable salt), ester, tautomer, prodrug, zwitterionic form, or stereoisomer thereof, wherein:
each X¹ is selected from hydrogen and deuterium, and each X¹ is the same;
each X² is selected from hydrogen and deuterium, and each X² is the same;
each X³ is selected from hydrogen and deuterium, and each X³ is the same;
each X⁴ is selected from hydrogen and deuterium, and each X⁴ is the same;
each X⁵ is selected from hydrogen and deuterium, and each X⁵ is the same; and
each X⁶ is selected from hydrogen and deuterium, and each X⁶ is the same,
provided that at least one X¹, X², X³, X⁴, X⁵, or X⁶ is deuterium.

In some embodiments, each X¹ is hydrogen. In some embodiments, each X¹ is deuterium.

In some embodiments, each X² is hydrogen. In some embodiments, each X² is deuterium.

In some embodiments, each X³ is hydrogen. In some embodiments, each X³ is deuterium.

In some embodiments, each X⁴ is hydrogen. In some embodiments, each X⁴ is deuterium.

In some embodiments, each X⁵ is hydrogen. In some embodiments, each X⁵ is deuterium.

In some embodiments, each X⁶ is hydrogen. In some embodiments, each X⁶ is deuterium.

In some embodiments, if each X¹ is hydrogen, each X³ is also hydrogen. In some embodiments, if each X¹ and each X³ is hydrogen, each X² is deuterium. In some embodiments, if each X¹ and each X³ is hydrogen, each X² and each X⁴ is deuterium.

In some embodiments, if each X² is hydrogen, each X⁴ is also hydrogen. In some embodiments, if each X² and each X⁴ is hydrogen, each X¹ is deuterium. In some embodiments, if each X² and each X⁴ is hydrogen, each X¹ and each X³ is deuterium.

In some embodiments, if each X¹ is deuterium, each X³ is also deuterium. In some embodiments, if each X¹ and each X³ is deuterium, each X² is hydrogen. In some embodiments, if each X¹ and each X³ is deuterium, each X² and each X⁴ is hydrogen.

In some embodiments, if each X² is deuterium, each X⁴ is also deuterium. In some embodiments, if each X² and each X⁴ is deuterium, each X¹ is hydrogen. In some embodiments, if each X² and each X⁴ is deuterium, each X¹ and each X³ is hydrogen.

In some embodiments, each X¹, X², X³, and X⁴ is hydrogen. In some embodiments, if each X¹, X², X³, and X⁴ is hydrogen, each X⁵ is deuterium. In some embodiments, if each X¹, X², X³, and X⁴ is hydrogen, each X⁶ is deuterium. In some embodiments, if each X¹, X², X³, and X⁴ is hydrogen, each X⁵ and each X⁶ is deuterium.

In some embodiments, the compound is a compound represented by any one of Formulas Ia, Ib, Ic, Id, Ie, and If:

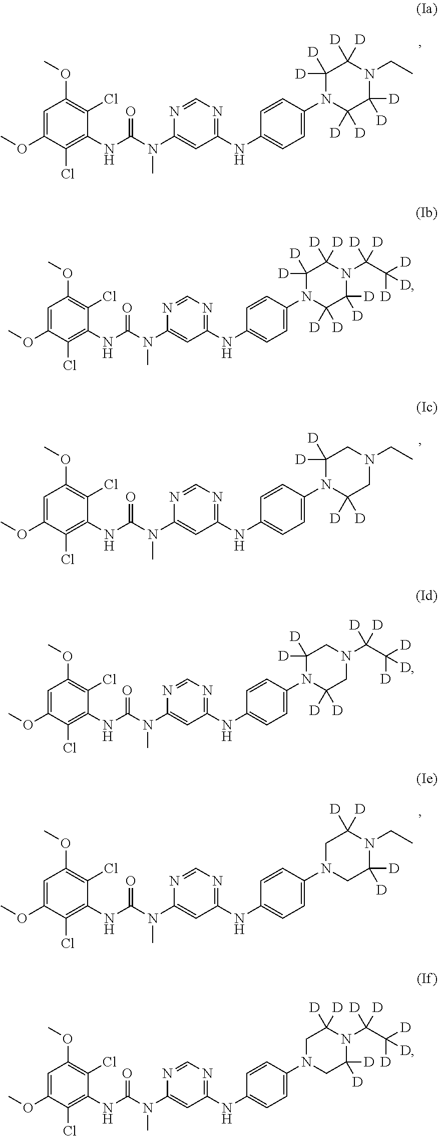

or a salt (e.g., pharmaceutically acceptable salt), ester, tautomer, prodrug, zwitterionic form, or stereoisomer thereof.

In some embodiments, the compound is a compound represented by Formula Ia, or a pharmaceutically acceptable salt thereof. In some embodiments, the compound is a compound represented by Formula Ia.

In some embodiments, the compound is a compound represented by Formula Ib, or a pharmaceutically acceptable salt thereof. In some embodiments, the compound is a compound represented by Formula Ib.

In some embodiments, the compound is a compound represented by Formula Ic, or a pharmaceutically acceptable salt thereof. In some embodiments, the compound is a compound represented by Formula Ic.

In some embodiments, the compound is a compound represented by Formula Id, or a pharmaceutically acceptable salt thereof. In some embodiments, the compound is a compound represented by Formula Id.

In some embodiments, the compound is a compound represented by Formula Ie, or a pharmaceutically acceptable salt thereof. In some embodiments, the compound is a compound represented by Formula Ic.

In some embodiments, the compound is a compound represented by Formula If, or a pharmaceutically acceptable salt thereof. In some embodiments, the compound is a compound represented by Formula If.

In some embodiments, for a compound according to any one of Formulas I, Ia, Ib, Ic, Id, Ie, and If, any atom not specifically designated as a particular isotope is meant to represent any stable isotope of that atom. In some embodiments, any hydrogen atom not designated as “D” (deuterium) is present at its natural isotopic abundance.

In some embodiments, for a compound according to any one of Formulas I, Ia, Ib, Ic, Id, Ie, and If, when a position is designated specifically as “D,” that position has at least about 50% incorporation of deuterium, such as at least about 60%, about 65%, about 70%, about 75%, about 80%, about 85%, about 90%, about 91%, about 92%, about 93%, about 94%, about 95%, about 96%, about 97%, about 98%, about 99%, or about 99.5% incorporation. In some embodiments, when a position is designated specifically as “D,” that position has at least about 90% incorporation of deuterium. In some embodiments, when a position is designated specifically as “D,” that position has at least about 95% incorporation of deuterium. In some embodiments, when a position is designated specifically as “D,” that position has at least about 97% incorporation of deuterium. In some embodiments, when a position is designated specifically as “D,” that position has at least about 99% incorporation of deuterium. In some embodiments, when a position is designated specifically as “D,” that position has at least about 99.5% incorporation of deuterium.

The compounds provided herein, including compounds according to any one of Formulas I, Ia, Ib, Ic, Id, Ie, and If, or a salt (e.g., pharmaceutically acceptable salt), ester, tautomer, prodrug, zwitterionic form, or stereoisomer thereof, can be synthesized according to one of the general routes outlined in the Synthetic Examples provided herein, or by various other methods generally known in the art.

Without wishing to be bound by theory, substitution of a compound with one or more deuterium atoms may impact the compound's metabolic properties. For example, replacement of one or more hydrogen atoms with a deuterium atom may slow the metabolism (e.g., CYP-mediated metabolism) of a compound and/or reduce the formation of one or more metabolites of the compound. Deuterium is generally regarded as a safe, stable, non-radioactive isotope of hydrogen that forms stronger bonds with carbon than hydrogen. Accordingly, in some instances, the increased strength of a given carbon-deuterium bond may favorably impact the absorption, distribution, metabolism, and/or excretion (ADME) properties of a compound, thereby providing a compound that may have improved efficacy, safety, and/or tolerability compared to the non-deuterated version. Moreover, as deuterium and hydrogen are approximately the same size and shape, replacement of a hydrogen atom by deuterium would generally not impact the biochemical potency and selectivity of a compound relative to a non-deuterated version.

### Compositions

The present disclosure also provides a composition (e.g., a pharmaceutical composition) comprising a compound provided herein (e.g., a compound of any one of Formulas I, Ia, Ib, Ic, Id, Ie, and If), or a salt (e.g., pharmaceutically acceptable salt), ester, tautomer, prodrug, zwitterionic form, or stereoisomer thereof. In some embodiments, a provided composition comprises a compound provided herein, or a pharmaceutically acceptable salt thereof. For example, the present disclosure provides a pharmaceutical composition comprising a compound provided herein (e.g., a compound of any one of Formulas I, Ia, Ib, Ic, Id, Ie, and If), or a salt, ester, tautomer, prodrug, zwitterionic form, or stereoisomer thereof, together with a pharmaceutically acceptable carrier. In some embodiments, a provided pharmaceutical composition comprises a compound provided herein or a pharmaceutically acceptable salt thereof, together with a pharmaceutically acceptable carrier.

In some embodiments, the pharmaceutical composition is formulated for oral administration. In some embodiments, the oral pharmaceutical formulation is selected from a tablet and a capsule.

In some embodiments, the pharmaceutical composition is formulated for parenteral administration. In some embodiments, the pharmaceutical composition is formulated for intravenous administration. In some embodiments, the pharmaceutical composition is formulated for subcutaneous administration.

While it may be possible for certain compounds provided herein (e.g., a compound of any one of Formulas I, Ia, Ib, Ic, Id, Ie, and If), or a salt, ester, tautomer, prodrug, zwitterionic form, or stereoisomer thereof, to be administered as the raw chemical, compounds may additionally or alternatively be provided in a pharmaceutical formulation. Accordingly, provided herein are pharmaceutical formulations which comprise one or more compounds disclosed herein (e.g., a compound of any one of Formulas I, Ia, Ib, Ic, Id, Ie, and If), or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration selected. Any of the well-known techniques, carriers, and excipients may be used as suitable and as understood in the art. The pharmaceutical compositions disclosed herein may be manufactured in any suitable manner known, e.g., by means of conventional mixing, dissolving, granulating, dragee-making, levigating, emulsifying, encapsulating, entrapping or compression processes.

A pharmaceutical formulation provided herein can be suitable for oral, parenteral (including subcutaneous, intradermal, intramuscular, intravenous, intraarticular, and intramedullary), intraperitoneal, transmucosal, transdermal, rectal, and topical (including dermal, buccal, sublingual, and intraocular) administration. The most suitable route may depend on, for example, the condition and disorder of the subject to which the pharmaceutical formulation will be administered. A pharmaceutical formulation can be provided in a unit dosage form. A pharmaceutical formulation can be prepared by any suitable method. A method of preparing a pharmaceutical formulation may comprise bringing a compound provided herein (e.g., a compound of any one of Formulas I, Ia, Ib, Ic, Id, Ie, and If), or a pharmaceutically acceptable salt, ester, amide, prodrug or solvate thereof (“active ingredient”) in contact with one or more pharmaceutically acceptable carriers (e.g., accessory ingredients). In general, the formulations are prepared by uniformly and intimately bringing into association the active ingredient with liquid carriers or finely divided solid carriers or both and then, if necessary, shaping the product into the desired formulation.

Pharmaceutical formulations of compounds provided herein (e.g., compounds of any one of Formulas I, Ia, Ib, Ic, Id, Ie, and If in any available form (e.g., salt, ester, tautomer, prodrug, zwitterionic form, stereoisomer etc.)) may be provided as discrete units. For example, a formulation suitable for oral administration may be provided as capsules, cachets, and/or tablets containing a predetermined amount of the compound in any suitable form (e.g., the active ingredient); as a solution or suspension in a solvent (e.g., aqueous or non-aqueous solvent); as an emulsion (e.g., an oil-in-water liquid emulsion or water-in-oil liquid emulsion); or as a powder or granules. The active ingredient may additionally or alternatively be provided as a bolus, electuary, or paste.

Pharmaceutical preparations suitable for oral administration include tablets, push-fit capsules made of gelatin, as well as soft, sealed capsules made of gelatin and a plasticizer, such as glycerol or sorbitol. Tablets may be made by, for example, compression or molding, optionally with one or more accessory ingredients, such as one or more pharmaceutically acceptable excipients. Compressed tablets may be prepared by, for example, compressing in a suitable machine the active ingredient in a free-flowing form such as a powder or granules, optionally mixed with binders, inert diluents, or lubricating, surface active or dispersing agents. Molded tablets may be made by, for example, molding in a suitable machine a mixture of the powdered compound moistened with an inert liquid diluent. The tablets may optionally be coated or scored and may be formulated to provide slow or controlled release of the active ingredient therein. All formulations for oral administration should be in dosages suitable for such administration. The push-fit capsules can contain the active ingredients in admixture with, for example, one or more fillers such as lactose, one or more binders such as one or more starches, and/or one or more lubricants such as talc or magnesium stearate and, optionally, one or more stabilizers. In soft capsules, the active compounds may be dissolved or suspended in suitable liquids, such as fatty oils, liquid paraffin, or liquid polyethylene glycols. Stabilizers and other elements may also be added. Dragee cores are provided with suitable coatings. For this purpose, concentrated sugar solutions may be used, which may optionally contain a gum, gelling agent, polymer, solvent, or combination thereof. Dyestuffs or pigments may be added to the tablets or dragee coatings for identification or to characterize different combinations of active compound doses.

A pharmaceutical composition comprising a compound provided herein (e.g., a compound of any one of Formulas I, Ia, Ib, Ic, Id, Ie, and If), or a form thereof (e.g., salt, ester, tautomer, prodrug, zwitterionic form, stereoisomer, etc.), may be formulated for parenteral administration by injection, e.g., by bolus injection or continuous infusion. Formulations for injection may be presented in unit dosage form, e.g., in ampoules, vials, or in multi-dose containers, with an added preservative. The compositions may take such forms as suspensions, solutions, or emulsions in oily or aqueous vehicles, and may contain formulating agents such as suspending, stabilizing, and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, prior (e.g., immediately prior) to use. Extemporaneous injection solutions and suspensions may be prepared from sterile powders, granules and tablets of the kind previously described.

A pharmaceutical composition comprising a compound provided herein (e.g., a compound of any one of Formulas I, Ia, Ib, Ic, Id, Ie, and If), or a form thereof (e.g., salt, ester, tautomer, prodrug, zwitterionic form, stereoisomer etc.), may be formulated as a solution for injection, which solution may be an aqueous or non-aqueous (oily) sterile solution and may comprise one or more antioxidants, thickening agents, suspending agents, buffers, solutes, and/or bacteriostats. The addition of one or more such additives may render the formulation isotonic with the blood of the intended recipient (e.g., subject or patient). Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described elsewhere herein, the compounds provided herein (e.g., compounds of any one of Formulas I, Ia, Ib, Ic, Id, Ie, and If in any suitable form (e.g., salt, ester, tautomer, prodrug, zwitterionic form, stereoisomer, etc.)), may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical composition comprising a compound provided herein (e.g., a compound of any one of Formulas I, Ia, Ib, Ic, Id, Ie, and If) or a form thereof (e.g., salt, ester, tautomer, prodrug, zwitterionic form, stereoisomer, etc.) that is suitable for buccal or sublingual administration may take the form of tablets, lozenges, pastilles, or gels. Such compositions may comprise the active ingredient in a flavored basis such as sucrose and acacia or tragacanth. A pharmaceutical composition comprising a compound provided herein or a form thereof (e.g., salt, ester, tautomer, prodrug, zwitterionic form, stereoisomer, etc.) that is suitable for rectal administration may be formulated as a suppository or retention enema and may comprise a medium such as, for example, cocoa butter, polyethylene glycol, or other glycerides.

Certain compounds provided herein (e.g., a compound of any one of Formulas I, Ia, Ib, Ic, Id, Ie, and If) or a form thereof (e.g., salt, ester, tautomer, prodrug, zwitterionic form, stereoisomer, etc.) may be formulated for non-systemic administration, such as topical administration. This includes the application of a compound disclosed herein, or a form thereof, externally to the epidermis or the buccal cavity and the instillation of such a compound, or a form thereof, into the ear, eye, and nose, such that the compound, or a form thereof, does not significantly enter the blood stream. In contrast, systemic administration refers to oral, intravenous, intraperitoneal, and intramuscular administration.

Formulations suitable for topical administration include liquid or semi-liquid preparations suitable for penetration through the skin to the site of inflammation such as gels, liniments, lotions, creams, ointments, or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds (e.g., compounds of any one of Formulas I, Ia, Ib, Ic, Id, Ie, and If) or forms thereof (e.g., salt, ester, tautomer, prodrug, zwitterionic form, stereoisomer, etc.) may be conveniently delivered from an insufflator, nebulizer pressurized packs, or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Alternatively, for administration by inhalation or insufflation, the compounds provided herein may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as described herein, or an appropriate fraction thereof, of the active ingredient (e.g., a compound provided herein (e.g., a compound of any one of Formulas I, Ia, Ib, Ic, Id, Ie, and If), or a salt, ester, tautomer, prodrug, zwitterionic form, or stereoisomer thereof).

It should be understood that in addition to the ingredients particularly described elsewhere herein, the formulations described herein may include other useful agents having regard to the type of formulation in question, for example those suitable for oral administration may include flavoring agents.

Compounds (e.g., compounds of any one of Formulas I, Ia, Ib, Ic, Id, Ie, and If) or forms thereof (e.g., salt, ester, tautomer, prodrug, zwitterionic form, stereoisomer, etc.) may be administered orally or via injection at a dose of from 0.001 to 500 mg/kg per day. Tablets or other forms of presentation provided in discrete units may conveniently contain an amount of one or more compounds which is effective at such dosage or as a multiple of the same, for instance, units containing 5 mg to 500 mg.

The amount of active ingredient that may be combined with the carrier materials to produce a single dosage form will vary depending upon the host treated and the particular mode of administration.

### Uses and Methods

The present disclosure also provides a method of modulating (e.g., inhibiting) an FGFR (e.g., FGFR1, FGFR2, FGFR3, and/or FGFR4, such as a mutant, rearranged, or fused FGFR) comprising contacting the FGFR with a compound provided herein (e.g., a compound of any one of Formulas I, Ia, Ib, Ic, Id, Ie, and If), or a salt, ester, tautomer, prodrug, zwitterionic form, or stereoisomer thereof. For example, the present disclosure may provide a method of altering a cell phenotype, cell proliferation, FGFR activity, biochemical output produced by FGFR, expression of FGFR, and/or binding of FGFR with a natural binding partner. Any such feature may be monitored and may be altered upon contacting an FGFR with a compound provided herein, or a form thereof. A method of modulating (e.g., inhibiting) FGFR (e.g., FGFR1, FGFR2, FGFR3, and/or FGFR4, such as a mutant, rearranged, or fused FGFR) may be a mode of treatment of a disease, disorder, or condition (e.g., a cancer or skeletal dysplasia, as described herein), a biological assay, a cellular assay, a biochemical assay, etc. In some embodiments, a method of modulating (e.g., inhibiting) an FGFR (e.g., FGFR1, FGFR2, FGFR3, and/or FGFR4, such as a mutant, rearranged, or fused FGFR) comprises contacting an FGFR with a compound provided herein, or a salt, ester, tautomer, zwitterionic form, or stereoisomer thereof. In some embodiments, contacting an FGFR with a compound provided herein, or a salt, ester, tautomer, zwitterionic form, or stereoisomer thereof, comprises incubating the FGFR with the compound or form thereof. In some embodiments, contacting an FGFR with a compound provided herein, or a salt, ester, tautomer, zwitterionic form, or stereoisomer thereof, comprises contacting a cell containing the FGFR with the compound or form thereof. In some embodiments, the cell is in a subject. In some embodiments, the subject is a human. In some embodiments, the subject is a human having a disease, disorder, or condition such as a cancer or skeletal dysplasia (e.g., as described herein). In some embodiments, the subject is less than 18 years of age (e.g., a pediatric subject) (e.g., as described herein).

The present disclosure also provides methods of treating a disease, disorder, or condition in a subject in need thereof using a compound provided herein, (e.g., a compound of any one of Formulas I, Ia, Ib, Ic, Id, Ie, and If), or a salt, ester, tautomer, prodrug, zwitterionic form, or stereoisomer thereof. For example, the present disclosure provides a method comprising providing (e.g., administering) to a subject (e.g., patient) in need thereof an effective amount of a compound provided herein (e.g., a compound of any one of Formulas I, Ia, Ib, Ic, Id, Ie, and If), or a salt, ester, tautomer, prodrug, zwitterionic form, or stereoisomer thereof. The present disclosure also provides methods of treating a disease, disorder, or condition in a subject in need thereof using a pharmaceutical composition comprising a compound provided herein, (e.g., a compound of any one of Formulas I, Ia, Ib, Ic, Id, Ie, and If), or a salt, ester, tautomer, prodrug, zwitterionic form, or stereoisomer thereof. For example, the present disclosure provides a method comprising providing (e.g., administering) to a subject (e.g., patient) in need thereof a pharmaceutical composition comprising an effective amount of a compound provided herein (e.g., a compound of any one of Formulas I, Ia, Ib, Ic, Id, Ie, and If), or a salt, ester, tautomer, prodrug, zwitterionic form, or stereoisomer thereof. In some embodiments, the subject is known to have (e.g., has previously been diagnosed with) a disease, disorder, or condition such as a cancer. The disease, disorder, or condition may be an FGFR-mediated disease (e.g., an FGFR1-, FGFR2-, FGFR3- and/or FGFR4 mediated disease), such as a cancer, skeletal dysplasia, or other disorder characterized by a mutant, rearranged, or fused FGFR. In some embodiments, the compound administered to the subject in need thereof according to the methods described herein is a compound described in an embodiment, example, figure, or table herein, or a stereoisomer or pharmaceutically acceptable salt thereof. In some embodiments, the subject is less than 18 years of age (e.g., a pediatric subject) (e.g., as described herein).

The present disclosure also provides a compound as provided herein (e.g., a compound of any one of Formulas I, Ia, Ib, Ic, Id, Ie, and If), or a salt, ester, tautomer, prodrug, zwitterionic form, or stereoisomer thereof, or a pharmaceutical composition comprising any of the foregoing compounds and a pharmaceutically acceptable excipient, for use as a medicament, such as a medicament for the treatment of a disease, disorder, or condition (e.g., a cancer or skeletal dysplasia). The present disclosure also provides a compound as provided herein (e.g., a compound of any one of Formulas I, Ia, Ib, Ic, Id, Ie, and If), or a salt, ester, tautomer, prodrug, zwitterionic form, or stereoisomer thereof, or a pharmaceutical composition comprising any of the foregoing compounds and a pharmaceutically acceptable excipient, for use in the manufacture of a medicament for the treatment of a disease, disorder, or condition (e.g., a cancer or skeletal dysplasia) in a subject in need thereof. In some embodiments, the subject is less than 18 years of age (e.g., a pediatric subject) (e.g., as described herein).

The present disclosure also provides the use of a compound provided herein (e.g., a compound of any one of Formulas I, Ia, Ib, Ic, Id, Ie, and If), or a salt, ester, tautomer, prodrug, zwitterionic form, or stereoisomer thereof, or a pharmaceutical composition comprising any of the foregoing compounds and a pharmaceutically acceptable excipient, for the treatment of a disease, disorder, or condition (e.g., a cancer or skeletal dysplasia, as described herein) in a subject in need thereof. In some embodiments, the subject is less than 18 years of age (e.g., a pediatric subject) (e.g., as described herein).

The present disclosure also provides the use of a compound provided herein (e.g., a compound of any one of Formulas I, Ia, Ib, Ic, Id, Ie, and If), or a salt, ester, tautomer, prodrug, zwitterionic form, or stereoisomer thereof, or a pharmaceutical composition comprising any of the foregoing compounds and a pharmaceutically acceptable excipient, in the manufacture of a medicament for treating a disease, disorder, or condition (e.g., a cancer or skeletal dysplasia, as described herein) in a subject in need thereof. In some embodiments, the subject is less than 18 years of age (e.g., a pediatric subject) (e.g., as described herein).

The present disclosure also provides a method of inhibiting an FGFR (e.g., FGFR1, FGFR2, FGFR3, and/or FGFR4, such as a mutant, rearranged, or fused FGFR) (e.g., in a subject in need thereof) comprising contacting the FGFR with a compound as provided herein (e.g., a compound of any one of Formulas I, Ia, Ib, Ic, Id, Ie, and If), or a salt, ester, tautomer, prodrug, zwitterionic form, or stereoisomer thereof, or a pharmaceutical composition comprising any of the foregoing compounds and a pharmaceutically acceptable excipient. In some embodiments, contacting an FGFR with a compound provided herein, or a salt, ester, tautomer, zwitterionic form, or stereoisomer thereof, comprises incubating the FGFR with the compound or form thereof. In some embodiments, contacting an FGFR with a compound provided herein, or a salt, ester, tautomer, zwitterionic form, or stereoisomer thereof, comprises contacting a cell containing the FGFR with the compound or form thereof. In some embodiments, the cell is in a subject. In some embodiments, the subject is a human. In some embodiments, the subject is a human having a disease, disorder, or condition such as a cancer or skeletal dysplasia (e.g., as described herein). In some embodiments, the subject is less than 18 years of age (e.g., a pediatric subject) (e.g., as described herein).

The present disclosure also provides a compound as provided herein (e.g., a compound of any one of Formulas I, Ia, Ib, Ic, Id, Ie, and If), or a salt, ester, tautomer, prodrug, zwitterionic form, or stereoisomer thereof, or a pharmaceutical composition comprising any of the foregoing compounds and a pharmaceutically acceptable excipient, for use in inhibiting FGFR (e.g., FGFR1, FGFR2, FGFR3, and/or FGFR4, such as a mutant, rearranged, or fused FGFR) (e.g., in a subject in need thereof). The present disclosure also provides a compound as provided herein (e.g., a compound of any one of Formulas I, Ia, Ib, Ic, Id, Ie, and If), or a salt, ester, tautomer, prodrug, zwitterionic form, or stereoisomer thereof, or a pharmaceutical composition comprising any of the foregoing compounds and a pharmaceutically acceptable excipient, for use in the manufacture of a medicament for inhibiting FGFR (e.g., FGFR1, FGFR2, FGFR3, and/or FGFR4, such as a mutant, rearranged, or fused FGFR) in a subject in need thereof. In some embodiments, the subject is less than 18 years of age (e.g., a pediatric subject) (e.g., as described herein).

The present disclosure also provides the use of a compound provided herein (e.g., a compound of any one of Formulas I, Ia, Ib, Ic, Id, Ie, and If), or a salt, ester, tautomer, prodrug, zwitterionic form, or stereoisomer thereof, or a pharmaceutical composition comprising any of the foregoing compounds and a pharmaceutically acceptable excipient, for inhibiting FGFR (e.g., FGFR1, FGFR2, FGFR3, and/or FGFR4, such as a mutant, rearranged, or fused FGFR) in a subject in need thereof. In some embodiments, the subject is less than 18 years of age (e.g., a pediatric subject) (e.g., as described herein).

The present disclosure also provides the use of a compound provided herein (e.g., a compound of any one of Formulas I, Ia, Ib, Ic, Id, Ie, and If), or a salt, ester, tautomer, prodrug, zwitterionic form, or stereoisomer thereof, or a pharmaceutical composition comprising any of the foregoing compounds and a pharmaceutically acceptable excipient, in the manufacture of a medicament for inhibiting FGFR (e.g., FGFR1, FGFR2, FGFR3, and/or FGFR4, such as a mutant, rearranged, or fused FGFR) in a subject in need thereof. In some embodiments, the subject is less than 18 years of age (e.g., a pediatric subject) (e.g., as described herein).

The present disclosure also provides a method comprising administering a therapeutically effective amount of a compound provided herein (e.g., a compound of any one of Formulas I, Ia, Ib, Ic, Id, Ie, and If), or a salt, ester, tautomer, prodrug, zwitterionic form, or stereoisomer thereof to a subject (e.g., patient) (e.g., a subject in need thereof), thereby ameliorating, reducing, eliminating, ceasing, delaying the progression of, or improving one or more symptoms of the subject, such as one or more symptoms of a disease, disorder, or condition (e.g., a cancer or skeletal dysplasia, as described herein). In some embodiments, the subject has a cancer, skeletal dysplasia, or other condition characterized by a mutant, rearranged, or fused FGFR (e.g., a mutant, rearranged, or fused FGFR1, FGFR2, FGFR3, and/or FGFR4). In some embodiments, the subject is less than 18 years of age (e.g., a pediatric subject) (e.g., as described herein).

In some embodiments, administering a therapeutically effective amount of a compound provided herein (e.g., a compound of any one of Formulas I, Ia, Ib, Ic, Id, Ie, and If), or a salt, ester, tautomer, prodrug, zwitterionic form, or stereoisomer thereof, slows or prevents growth of a tumor. In some embodiments, administering a therapeutically effective amount of a compound provided herein (e.g., a compound of any one of Formulas I, Ia, Ib, Ic, Id, Ie, and If), or a salt, ester, tautomer, prodrug, zwitterionic form, or stereoisomer thereof, results in shrinkage of a tumor (e.g., tumor regression). In some embodiments, administering a therapeutically effective amount of a compound provided herein (e.g., a compound of any one of Formulas I, Ia, Ib, Ic, Id, Ie, and If), or a salt, ester, tautomer, prodrug, zwitterionic form, or stereoisomer thereof, results in at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% regression of a tumor, such as for a period of one or more weeks (e.g., at least about 1, 2, 3, 4, 5, 6, 7, 8, 9, 10, 11, 12, or more weeks), a period of one or more months (e.g., at least about 1, 2, 3, 4, 5, 6, 7, 8, 9, 10, 11, 12, or more months), or a period of one or more years (e.g., at least about 1, 2, 3, or more years). In some embodiments, administering a therapeutically effective amount of a compound provided herein (e.g., a compound of any one of Formulas I, Ia, Ib, Ic, Id, Ie, and If), or a salt, ester, tautomer, prodrug, zwitterionic form, or stereoisomer thereof, stabilizes a tumor. In some embodiments, administering a therapeutically effective amount of a compound provided herein (e.g., a compound of any one of Formulas I, Ia, Ib, Ic, Id, Ie, and If), or a salt, ester, tautomer, prodrug, zwitterionic form, or stereoisomer thereof, stabilizes a tumor for a period of one or more weeks (e.g., at least about 1, 2, 3, 4, 5, 6, 7, 8, 9, 10, 11, 12, or more weeks), a period of one or more months (e.g., at least about 1, 2, 3, 4, 5, 6, 7, 8, 9, 10, 11, 12, or more months), or a period of one or more years (e.g., at least about 1, 2, 3, or more years). In some embodiments, the subject has a cancer characterized by a mutant, rearranged, or fused FGFR (e.g., a mutant, rearranged, or fused FGFR1, FGFR2, FGFR3, and/or FGFR4).

In some embodiments of any of the methods, uses, and medicaments provided herein, the disease, disorder, or condition is a cancer. In some embodiments of any of the methods, uses, and medicaments provided herein, the cancer is pancreatic cancer (e.g., pancreatic ductal adenocarcinoma), lung cancer (e.g., non-small cell lung cancer), colorectal cancer (CRC), endometrial cancer, uterine carcinosarcoma, Ewing sarcoma, osteosarcoma, Rhabdomyosarcoma, adrenocortical carcinoma, neuroblastoma, Wilm tumor, retinoblastoma, skin cancer, breast cancer, prostate cancer, head and neck cancer, bladder cancer, salivary gland cancer, ovarian cancer, hematological cancer, phosphaturic mesenchymal tumor, sarcoma, epithelial cancer, cervix cancer, urothelial carcinoma, cholangiocarcinoma, glioblastoma, glioma, melanoma, or a myeloma. In some embodiments, the cancer is or comprises a solid tumor.

In some embodiments of any of the methods, uses, and medicaments provided herein, the disease, disorder, or condition is a skeletal dysplasia. In some embodiments, the skeletal dysplasia is selected from the group consisting of achondroplasia, hypochondroplasia, thanatophoric dysplasia, osteogenesis imperfecta, campomelic dysplasia, achnodrogenesis, severe achondroplasia with developmental delay and acanthosis nigricans (SADDAN), Muenke syndrome, and Crouzon syndrome (e.g., Crouzon syndrome acanthosis nigricans). In some embodiments, the skeletal dysplasia is selected from the group consisting of achondroplasia, hypochondroplasia, thanatophoric dysplasia, and severe achondroplasia with developmental delay and acanthosis nigricans (SADDAN). In some embodiments, the skeletal dysplasia is achondroplasia or hypochondroplasia. In some embodiments, the skeletal dysplasia is achondroplasia. In some embodiments, the skeletal dysplasia is hypochondroplasia.

In some embodiments, the disease, disorder, or condition is a congenital malformation of the musculoskeletal system. In some embodiments, the disease, disorder, or condition is selected from the group consisting of craniosynostosis, Kallmann syndrome, hypogonadotropic hypogonadism, cleft palate and other craniofacial defects, scoliosis, Type 1 or classic Pfeiffer syndrome, osteoglophonic dysplasia, craniosynostosis, mandibular progrnathism, hypertelorism, brachydactyly, inter-phalangeal joint fusion, Jackson-Weiss syndrome, Antley-Bixler syndrome, Apert syndrome, Crouzon syndrome, and trigonocephaly.

### Administration and Combination Therapy

The compounds provided herein (e.g., compounds of any one of Formulas I, Ia, Ib, Ic, Id, Ie, and If) and forms thereof (e.g., salt, ester, tautomer, prodrug, zwitterionic form, stereoisomer, etc.), or compositions (e.g., pharmaceutical compositions) comprising the same, can be administered in various modes (e.g., orally, topically, or by injection). The amount of active ingredient (e.g., a compound provided herein in any suitable form thereof) administered to a subject (e.g., patient) will be the responsibility of an attendant medical provider. The specific dose level for a given subject (e.g., patient) will depend on a variety of factors including, for example, the activity of the active ingredient administered; the physical attributes of the subject (e.g., age, weight, height, body mass index, general health, co-morbidities, sex, etc.); other characteristics of the subject (e.g., diet, level of exercise, national origin, ethnicity, etc.); time of administration; route of administration; rate of excretion; drug combination; the disease, disorder, or condition being treated; and the severity of the disease, disorder, or condition being treated.

In some embodiments, a compound provided herein (e.g., a compound of any one of Formulas I, la, Ib, Ic, Id, Ie, and If), or a form thereof (e.g., salt, ester, tautomer, prodrug, zwitterionic form, stereoisomer, etc.) is administered in combination with an additional agent, such as an additional therapeutic agent. For example, if a subject experiences a side effect such as hypertension upon receiving a compound provided herein, or a form thereof, it may be appropriate to administer an additional agent that is effective in managing the side effect, such as an anti-hypertensive agent. In another example, the therapeutic effectiveness of a compound provided herein (e.g., a compound of any one of Formulas I, Ia, Ib, Ic, Id, Ie, and If), or a form thereof, may be enhanced by administration of an adjuvant, which adjuvant may itself have only minimal therapeutic benefit, but in combination with another therapeutic agent may provide an enhanced overall therapeutic benefit to a subject. In a further example, the therapeutic benefit of a compound provided herein (e.g., a compound of any one of Formulas I, Ia, Ib, Ic, Id, Ie, and If), or a form thereof, may be enhanced by administration of the compound, or a form thereof, and an additional agent (which may comprise an additional therapeutic regimen) that also provides a therapeutic benefit. For example, a compound provided herein (e.g., a compound of any one of Formulas I, Ia, Ib, Ic, Id, Ie, and If), or a form thereof, may be administered in combination with an additional agent that may be effective in the treatment of a disease, disorder, or condition such as a cancer. Generally, the combination of a compound provided herein (e.g., a compound of any one of Formulas I, Ia, Ib, Ic, Id, Ie, and If), or a form thereof, and one or more additional agents (e.g., therapeutic agents) may enhance the overall benefit experienced by the subject upon either component individually. In some embodiments, the effect may be additive. In some embodiments, the effect may be synergistic.

In some embodiments, a compound provided herein (e.g., a compound of any one of Formulas I, la, Ib, Ic, Id, Ie, and If), or a form thereof (e.g., salt, ester, tautomer, prodrug, zwitterionic form, stereoisomer, etc.) is administered in combination with an anti-cancer agent (e.g., chemotherapeutic agent). An anti-cancer agent may be, for example, an alkylating agent, an antimitotic, a checkpoint inhibitor, an anti-metabolite, a plant alkaloid, a terpenoid, a cytotoxic agent, an antibiotic, a topoisomerase inhibitor, an aromatase inhibitor, an angiogenesis inhibitor, an anti-steroid, an anti-androgen, an mTOR inhibitor, monoclonal antibodies, or a tyrosine kinase inhibitor.

Two or more therapeutic agents, one of which is a compound provided herein (e.g., a compound of any one of Formulas I, Ia, Ib, Ic, Id, Ie, and If) or a form thereof, may be administered in any order or may be administered simultaneously. If administered simultaneously, the multiple therapeutic agents may be provided in a single, unified form, or in multiple forms (such as, for example, as a single pill or as two separate pills). One of the therapeutic agents may be given in multiple doses, or both may be given as multiple doses. If not administered simultaneously, the timing between the multiple doses may be any duration of time ranging from a few minutes to four weeks.

Accordingly, in another aspect, the present disclosure provides a method for treating a disease, disorder, or condition (e.g., a cancer) in a subject (e.g., a human or animal subject) in need of such treatment comprising administering to the subject an amount of a compound provided herein (e.g., a compound of any one of Formulas I, Ia, Ib, Ic, Id, Ie, and If), or a form thereof (e.g., salt, ester, tautomer, prodrug, zwitterionic form, stereoisomer, etc.), in combination with at least one additional agent for the treatment of the disease, disorder, or condition. In a related aspect, the present disclosure provides a composition (e.g., pharmaceutical composition) comprising a compound provided herein (e.g., a compound of any one of Formulas I, Ia, Ib, Ic, Id, Ie, and If), or a form thereof (e.g., salt, ester, tautomer, prodrug, zwitterionic form, stereoisomer, etc.), and at least one additional agent for use in the treatment of a disease, disorder, or condition (e.g., a cancer or skeletal dysplasia, as described herein).

In some embodiments, a method provided herein is used to treat a disease, disorder, or condition (e.g., a cancer or skeletal dysplasia) comprising administering to a subject in need thereof a therapeutically effective amount of a compound of any one of Formulas I, Ia, Ib, Ic, Id, Ie, and If or a pharmaceutically acceptable salt thereof. In some embodiments, the disease, disorder, or condition is a cancer that has developed a resistance to one or more chemotherapeutic drugs and/or ionizing radiation. In some embodiments, the disease, disorder, or condition is a cancer that has developed a resistance to one or more chemotherapeutic drugs and/or ionizing radiation.

The compounds, compositions, and methods disclosed herein are useful for the treatment of a disease, disorder, or condition, such as a cancer or skeletal dysplasia. In certain embodiments, the disease is one of dysregulated cellular proliferation, including cancer. The cancer may be hormone-dependent or hormone-resistant, such as in the case of breast cancers. In certain embodiments, the cancer is or comprises a solid tumor. In other embodiments, the cancer is a lymphoma or leukemia. In certain embodiments, the cancer is a drug resistant phenotype of a cancer disclosed herein or otherwise known. Tumor invasion, tumor growth, tumor metastasis, and angiogenesis may also be treated using the compositions and methods disclosed herein. In some embodiments, the compounds, compositions, and methods provided herein are also useful in the treatment of precancerous neoplasias.

Cancers that may be treated by the methods disclosed herein include, but are not limited to, pancreatic cancer, colon cancer, rectal cancer, colorectal cancer, breast cancer, ovarian cancer, endometrial cancer, lung cancer, and prostate cancer; cancers of the oral cavity and pharynx (lip, tongue, mouth, larynx, pharynx), esophagus, stomach, small intestine, large intestine, colon, rectum, liver and biliary passages; pancreas, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; and thyroid and other endocrine glands. The term “cancer” also encompasses cancers that do not necessarily form solid tumors, including Hodgkin's disease, non-Hodgkin's lymphomas, multiple myeloma, and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML),) and lymphomas including lymphocytic, granulocytic and monocytic lymphomas. Additional types of cancers which may be treated using the compounds and methods provided herein include, but are not limited to, adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor.

In some embodiments, the compounds, compositions, and methods provided herein are useful in the prevention and/or reduction of tumor invasion, growth, and/or metastasis.

The compounds, compositions, and methods provided herein may be useful in the treatment of humans as well as in the veterinary treatment of non-human animals including companion animals, exotic animals, and farm animals (e.g., as described herein), including mammals, rodents, and the like. For example, the compounds, compositions, and methods provided herein may be useful in the treatment of horses, dogs, or cats.

### EXAMPLES

### Example 1: Synthesis of 3-(2,6-dichloro-3,5-dimethoxy-phenyl)-1-{6-4-(4-ethyl-piperazin-1-yl)-phenylaminol-pyrimidin-4-yl}-1-methyl-urea (infigratinib)

### Step A: Synthesis of N-4-(4-ethyl-piperazin-1-yl)-phenyl)-N′-methyl-pyrimidine-4,6-diamine

A mixture of 4-(4-ethylpiperazin-1-yl)-aniline (1 g, 4.88 mmol), (6-chloro-pyrimidin-4-yl)-methyl-amine (1.81 g. 12.68 mmol. 1.3 eq.), and 4N HCl in dioxane (15 mL) is heated in a sealed tube to 150° C. for 5 hours. The reaction mixture is concentrated, diluted with dichloromethane (DCM) and a saturated aqueous solution of sodium bicarbonate. The aqueous layer is separated and extracted with DCM. The organic phase is washed with brine, dried (sodium sulfate), filtered and concentrated. Purification of the residue by silica gel column chromatography (DCM/MeOH, 93:7) followed by trituration in diethyl ether affords the title compound as a white solid: ESI-MS: 313.2 [MH]⁺; tR=1.10 min (gradient J); TLC: Rr=0.21 (DCM/MeOH, 93:7).

### Step B: Synthesis of 4-(4-ethylpiperazin-1-yl)-aniline

A suspension of 1-ethyl-4-(4-nitro-phenyl)-piperazine (6.2 g, 26.35 mmol) and Raney Nickel (2 g) in MeOH (120 mL) is stirred for 7 hours at RT, under a hydrogen atmosphere. The reaction mixture is filtered through a pad of celite and concentrated to afford 5.3 g of the title compound as a violet solid: ESI-MS: 206.1 [MH]⁺; TLC: Rr=0.15 (DCM/MeOH+1% NH_{3 aq}, 9:1).

### Step C: Synthesis of 1-ethyl-4-(4-nitro-phenyl)-piperazine

A mixture of 1-bromo-4-nitrobenzene (6 g, 29.7 mmol) and 1-ethylpiperazine (7.6 mL, 59.4 mmol, 2 eq.) is heated to 80° C. for 15 hours. After cooling to RT, the reaction mixture is diluted with water and DCM/MeOH, 9:1. The aqueous layer is separated and extracted with DCM/MeOH, 9:1. The organic phase is washed with brine, dried (sodium sulfate), filtered and concentrated. Purification of the residue by silica gel column chromatography (DCM/MeOH+1% NH_{aq}. 9:1) affords 6.2 g of the title compound as a yellow solid: ESI-MS: 236.0 [MH]⁺; tR=2.35 min (purity: 100%, gradient J); TLC: Rr=0.50 (DCM/MeOH+1% NH_{3 aq}, 9:1).

### Step D: Synthesis of (6-chloro-pyrimidin-4-yl)-methyl-amine

This material was prepared by a modified procedure published in the literature (J. Appl. Chem. 1955, 5, 358): To a suspension of commercially available 4,6-dichloropyrimidine (20 g, 131.6 mmol, 1.0 eq.) in isopropanol (60 mL) is added 33% methylamine in ethanol (40.1 mL, 328.9 mmol, 2.5 eq.) at such a rate that the internal temperature does not rise above 50° C. After completion of the addition the reaction mixture was stirred for 1 hour at room temperature.

Then, water (50 mL) is added and the suspension formed is chilled in an ice bath to 5° C. The precipitated product is filtered off, washed with cold isopropanol/water 2:1 (45 mL) and water. The collected material is vacuum dried over night at 45° C. to afford the title compound as colorless powder: t_{R}=3.57 min (purity: >99%, gradient A), ESI-MS: 144.3/146.2 [MH]⁺.

### Step E: Synthesis of 3-(2,6-dichloro-3,5-dimethoxy-phenyl)-1-{6-4-(4-ethyl-piperazin-1-yl)-phenylaminol-pyrimidin-4-yB-1-methyl-urea

The title compound was prepared by adding 2,6-dichloro-3,5-dimethoxyphenyl-isocyanate (1.25 eq.) to a solution of N-4-(4-ethyl-piperazin-1-yl)-phenyl)-N′-methyl-pyrimidine-4,6-diamine (2.39 g. 7.7 mmol, 1 eq.) in toluene and stirring the reaction mixture for 1.5 hours at reflux. Purification of the crude product by silica gel column chromatography (DCM/MeOH+1% NH3^{aq}, 95:5) affords the title compound as a white solid: ESI-MS: 560.0/561.9 [MH]⁺; te=3.54 min (purity: 100%, gradient J); TLC: Rr=0.28 (DCM/MeOH+1% NH₃ ^{aq}, 95:5). Analysis: C26H31N7O3Cl2, calc. C, 55.72%; H, 5.57%; N, 17.49%; O, 8.56%; Cl, 12.65%. Found C, 55.96%; H, 5.84%; N, 17.17%; O, 8.46%; Cl, 12.57%.

### Example 2: Synthesis of the Monophosphate Salt Form A of 3-(2,6-dichloro-3,5-dimethoxy-phenyl)-1-{6-4-(4-ethyl-piperazin-1-yl)-phenylaminol-pyrimidin-4-yl}-1-methyl-urea (BGJ398)

To a round bottom flask was added 3-(2,6-dichloro-3,5-dimethoxyphenyl)-1-(6-4-(4-ethylpiperazin-1-yl)phenylaminol-pyrimidine-4-yl)-1-methyl-urea (134 g, 240 mmol) and isopropanol (IPA) (2000 mb). The suspension was stirred and heated to 50° C. and a solution of phosphoric acid (73.5 g. 750 mmol) in water (2000 mb) added to it portions. The mixture was stirred at 60° C. for 30 minutes and filtered through a polypropylene pad. The pad was washed with warm IPA/water (1:1, 200 mb) and the filtrates were combined. To this clear solution, IPA (6000 mb) was added and the mixture was stirred under reflux for 20 minutes, cooled slowly to room temperature (25° C.), and stirred for 24 hours. The white salt product was collected by filtration, washed with IPA (2×500 mb) and dried in the oven at 60° C. under reduced pressure for two days to provide the anhydrous crystalline monophosphate salt (110 g). Yield 70%. Purity>98% by HPLC. Analysis: C26H34N7O7Cl2P, calc. C, 47.42%; H, 5.20%; N, 14.89%; O, 17.01%; Cl, 10.77%; P, 4.70%. Found C, 47.40%; H, 5.11%; N, 14.71%; O, 17.18%; Cl, 10.73%; P, 4.87%.

### Example 3: Biological Activity of Compounds

The efficacy of compounds provided herein as inhibitors of FGFRs including FGFR1, FGFR2, FGFR3, and FGFR4 can be evaluated as described in, for example U.S. Pat. No. 8,552,002; International Patent Application No. PCT/IB2011/003253; and International Patent Application No. PCT/US2021/065679, each of which are herein incorporated by reference in their entireties.

In one method, kinase-tagged T7 phage strains are prepared in E. coli host derived from, e.g., the BL21 strain. E. coli are grown and infected with T7 phage and incubated at elevated temperature with shaking until lysis. Lysates are centrifuged and filtered to remove cell debris. Streptavidin-coated magnetic beads are treated with biotinylated ligands at room temperature to generate affinity resins for kinase assays. Unbound ligand is removed and beads are washed with excess biotin. Binding reactions are assembled by combining kinases, liganded affinity beads, and test compounds in a binding buffer. Binding constants (Kds) can be determined using a dilution series. Reactions are performed in a standard well plate. Assay plates are incubated at room temperature with agitation and affinity beads are washed with a wash buffer before resuspension in an elution buffer and subsequent incubation. Kinase concentrations in eluates can be measured using quantitative PCR. Binding constants can be calculated using a standard dose-response curve using the Hill equation. Binding constants can be determined for each of FGFR1, FGFR2, FGFR3, and FGFR4 and, optionally, one or more mutant variants thereof, such as FGFR3 V555M. Results can be compared to those obtained for infigratinib.

Cell viability assays can also be performed to assess IC50 values for FGFR1, FGFR2, FGFR3, and/or FGFR4. Selectivity ratios for, e.g., FGFR3 relative to other FGFRs can also be assessed to determine the selectivity of the compounds provided herein for different FGFRs. Results can be compared to those obtained for infigratinib.

### INCORPORATION BY REFERENCE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

It should be understood from the foregoing that, while particular implementations have been illustrated and described, various modifications may be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations, or relative proportions set forth herein which depend upon a variety of conditions and variables. Various modifications in form and detail of the embodiments of the invention will be apparent to a person skilled in the art. It is therefore contemplated that the invention shall also cover any such modifications, variations, and equivalents. It is intended that the following claims define the scope of the invention and that the methods and structures within the scope of these claims and their equivalents be covered thereby.

1. A compound according to Formula I: 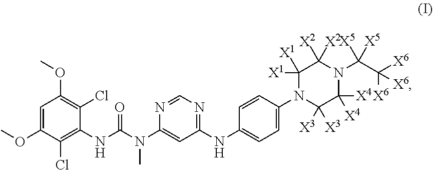
or a salt (e.g., pharmaceutically acceptable salt) thereof, wherein:
each X¹ is selected from hydrogen and deuterium, and each X¹ is the same;
each X² is selected from hydrogen and deuterium, and each X² is the same;
each X³ is selected from hydrogen and deuterium, and each X³ is the same;
each X⁴ is selected from hydrogen and deuterium, and each X⁴ is the same;
each X⁵ is selected from hydrogen and deuterium, and each X⁵ is the same; and
each X⁶ is selected from hydrogen and deuterium, and each X⁶ is the same,
provided that at least one X¹, X², X³, X⁴, X⁵, or X⁶ is deuterium.

2.-94. (canceled)
